(19) United States
(12) Patent Application Publication
(10) Pub. No.: US 2017/0197053 A1
(43) Pub. Date: Jul. 13, 2017

(54) TRANSORAL PROCEDURE MASK

(71) Applicant: Novlet Marvalyn Fenwick, Milton, DE (US)

(72) Inventor: Novlet Marvalyn Fenwick, Milton, DE (US)

(21) Appl. No.: 14/989,875

(22) Filed: Jan. 7, 2016

(51) Int. Cl. A61M 16/06; A61B 17/24; A61M 16/08

(52) U.S. Cl. CPC A61M 16/0683; A61M 16/0875; A61M 16/085; A61B 17/24; A61M 16/0605; A61M 2202/0208; A61M 2230/432

(57) ABSTRACT

The system of the preferred embodiments includes a mask shell, where an opening of at least one square inch is located in the mask over the user's mouth, where the mask shell is designed to fit over the user's mouth and nose; a strap connected to either lateral side of the mask shell and adapted to go at least one of behind the user's head and around the user's ears to pull the mask onto the user's face; an oxygen supply tube connected to the side of the mask shell over the user's cheek, and in fluid communication with the inside of the interior cavity of the mask shell; a second tube connected within a half inch of the oxygen supply tube, wherein the distal end of the second tube connects to a carbon dioxide sensor, wherein the second tube is adapted to convey exhaled gas to the carbon dioxide sensor. The transoral procedure mask of the first preferred embodiments is preferably designed to allow simultaneous delivery of high flow rates of oxygen to a patient's nose and mouth while also allowing access into the patients mouth of one or more of instruments and tools, while not having nasal prongs which can lead to nasal burning in patients at high flowrates, and while having an integrated tube for connection to a carbon dioxide partial pressure sensor to provide constant and convenient oxygenation monitoring. The system of the first preferred embodiments may, however, be used for any suitable reason.

### RELATED APPLICATION

This application is a continuation-in-part of, and claims the benefit under 35 U.S.C. 121 of, co-pending U.S. Original application Ser. No. 14/657,522 with first named inventor Novlet Marvalyn Fenwick on 13 Mar. 2015 and entitled “OXYGEN DELIVERY MASK FOR TRANSORAL MEDICAL PROCEDURES,” hereby incorporated in its entirety by reference.

### BRIEF DESCRIPTION OF THE FIGURES

FIG. 1 is a schematic representation of a variation of the system of the preferred embodiments with the removable perforated cover in place over the opening.

FIG. 2 is a schematic representation of a variation of the system of the preferred embodiments with the removable perforated cover removed.

### DESCRIPTION OF THE PREFERRED EMBODIMENTS

The following description of the preferred embodiments of the invention is intended to enable someone skilled in the prior art to make and use this invention, but is not intended to limit the invention to these preferred embodiments.

### 1. First Preferred Embodiment

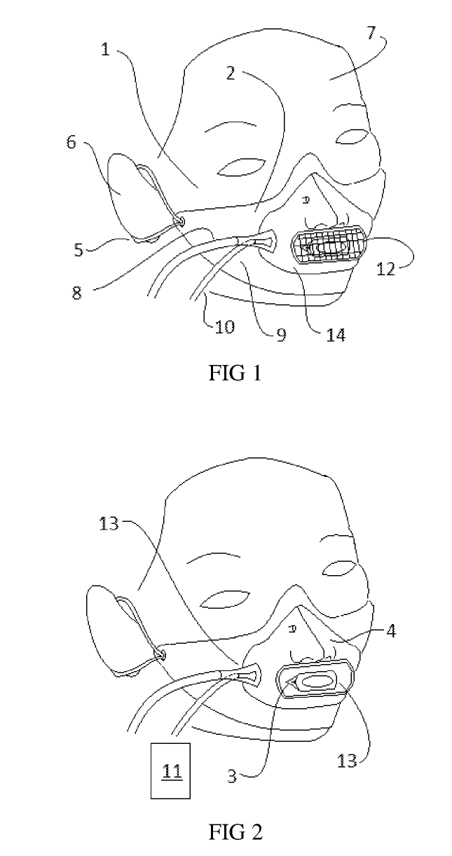

As shown in FIG. 1, the system of the preferred embodiments includes a mask shell 2, where an opening of at least one square inch is located in the mask over the user's mouth 3, where the mask shell 2 is designed to fit over the user's mouth 3 and nose 4; a strap 5 connected to either lateral side of the mask shell 2 and adapted to go at least one of behind the user's head and around the user's ears 6 to pull the mask onto the user's face 7; an oxygen supply tube 8 connected to the side of the mask shell 2 over the user's cheek 9, and in fluid communication with the inside of the interior cavity of the mask shell 2; a second tube 10 connected within a half inch of the oxygen supply tube 8, wherein the distal end of the second tube 10 connects to a carbon dioxide sensor 11, wherein the second tube 10 is adapted to convey exhaled gas to the carbon dioxide sensor 11. The transoral procedure mask 1 of the first preferred embodiments is preferably designed to allow simultaneous delivery of high flow rates of oxygen to a patient's nose 4 and mouth 3 while also allowing access into the patients mouth 3 of one or more of instruments and tools, while not having nasal prongs which can lead to nasal burning in patients at high flowrates, and while having an integrated tube for connection to a carbon dioxide partial pressure sensor to provide constant and convenient oxygenation monitoring. The system of the first preferred embodiments may, however, be used for any suitable reason.

As shown in FIG. 1, the transoral procedure mask 1 preferably includes a removable perforated cover 12 which removably attaches to cover the opening over the user's mouth 3 and includes at least two perforations. In a preferred variation, the perforated cover only has two or more small slits and does not allow significant fluid flow through the cover. In a preferred variation, the removable perforated cover 12 snap fits to the rim surrounding the opening over the user's mouth 3. In one preferred variation, the removable perforated cover 12 is constructed of at least one of polymer and metal screen. In another variation, the removable perforated cover 12 is constructed of a perforated piece of polymer with a plurality of openings created in one of a rigid piece of polymer and a semi-rigid piece of polymer. As shown in FIG. 2, the transoral procedure mask 1 can be removed from the opening in the mask shell 2, allowing access to the user's mouth 3 for at least one of tools and instruments. There may, however, be any suitable cover for the opening. There may, however, be no cover whatsoever.

As shown in FIG. 1, the transoral procedure mask 1 may further include a hollow conical protrusion 13 attached to the side of the mask shell 2, so that the broader end of the hollow conical protrusion 13 is attached to the side of the mask shell 2, and the apex of the hollow conical protrusion 13 is attached to a tube. The interior of the hollow conical protrusion 13 is in fluid communication with the tube and with the inside of the mask shell 2. The hollow conical protrusion 13 preferably expands and distributes the oxygen delivered thorough the tube and into the interior of the mask shell 2. The hollow conical protrusion 13 preferably entrains oxygen around the user's mouth 3 and allows the user to also inhale the oxygen through their nose 4. In another preferred variation, a second tube 10 is attached to the side of the hollow conical protrusion 13 such that the second tube 10 is in fluid communication with the interior of the hollow conical protrusion 13. In one variation of this variation, the second tube 10 is attached to the hollow conical protrusion 13 by passing it through the wall of the hollow conical protrusion 13. In a variation of this variation, a conical diffuser may be fitted to the end of the second tube 10 inside the hollow conical protrusion 13, where the conical diffuser is designed to fit inside the hollow conical protrusion 13, where the conical diffuser is further designed to help exhaled gases enter the second tube 10. Preferably the distal end of the second tube 10 is coupled in fluid communication to a carbon dioxide sensor 11. Preferably the carbon dioxide sensor 11 is designed to measure the partial pressure of carbon dioxide in the gases the user exhales. In this variation, the carbon dioxide sensor 11 preferably allows the oxygenation level of the user to be measured in order to monitor the proper oxygenation of the patient throughout a transoral procedure. There may, however, be any suitable attachment for the oxygen inlet tube. There may, however, be any suitable attachment for the second tube 10. There may, however, be no diffuser attached to the second tube 10 whatsoever.

As shown in FIG. 1, in one preferred variation a bite block may be attached to the face 7 mask shell 2 near the periphery of the opening. The bite block preferably includes an passageway at least 0.5 square inches in cross-sectional area passing through the bite block. The bite block and passageway preferably are designed to allow access to the user's mouth 3 for at least one of tools and instruments, allowing continuous access into a patient's mouth 3 during a transoral procedure and preventing the patients mouth 3 from fully closing.

The face 7 mask shell 2 is preferably designed to guide at least one of inlet oxygen and inlet oxygenated gas from the tube around the user's mouth 3 and into the user's nasal passageway. In prior art devices, nasal prongs deliver oxygen and this can directly lead to patient discomfort, and at high oxygenation rates it can also lead to burning sensations in the patient's nasal cavity. Furthermore, prior art devices do not allow convenient placement and attachment of oxygen delivery capable of successfully delivering high oxygen flow rates, second tube 10s used for monitoring exhaled carbon dioxide, and simultaneous access into the patient's mouth 3 to carry out a transoral procedure. The lack of such a device in prior art makes such procedures difficult, often requires multiple devices to be held or otherwise positioned in impromptu methods near the patient's mouth 3 and nose 4, and in some cases endangers patients by making proper oxygenation and measurement of exhaled gases difficult. The transoral procedure mask 1 may, however, be used in any suitable way and for any suitable purpose.

In a preferred variation, ventilation holes are included in the mask shell 2. In this variation, the ventilation holes preferably prevent the accumulation of carbon dioxide gas. There may, however, be no ventilation holes.

As shown in FIG. 1, in a preferred variation there may be at least one vent hole either near the bridge of the patient's nose 4, or laterally beside the patient's nose 4. This vent hole preferably encourages distribution of at least one of inlet oxygen and inlet oxygenated gas, and helps ensure that the gas is distributed to the user's nostrils as well as around the user's mouth 3.

In one preferred variation, the face 7 mask shell 2 includes two sections: a middle section 14 surrounding the user's mouth 3 and nose 4, where the middle section 14 is constructed of a rigid polymer, and where in one variation the hollow conical protrusion 13 is attached to the middle section 14 of the mask; and an outer section attached to the outer periphery of the middle section 14 and composed of a pliable polymer designed to better fit against and seal to a user's face 7.

As a person skilled in the art will recognize from the previous detailed description and from the figures and claims, modifications and changes can be made to the preferred embodiments of the invention without departing from the scope of this invention defined in the following claims.

I claim:

1) A transoral procedure mask comprising: a mask shell, wherein an opening of at least one square inch is located in the mask over the user's mouth, wherein the mask shell is designed to fit over the user's mouth and nose; a strap connected to either lateral side of the mask shell and adapted to go behind at least one of the user's head and the user's ears to pull the mask onto the user's face; an oxygen supply tube connected to the side of the mask shell over the user's cheek, and in fluid communication with the inside of the interior cavity of the mask shell; a second tube connected within a half inch of the oxygen supply tube, wherein the distal end of the second tube connects to a carbon dioxide sensor, wherein the second tube is adapted to convey exhaled gas to the carbon dioxide sensor.

2) The transoral procedure mask of claim 1, further comprising a removable perforated cover adapted to be removably attached to cover the opening, wherein the removable perforated cover comprises at least two perforations.

3) The transoral procedure mask of claim 2, wherein the removable perforated cover is adapted to snap fit to the rim of the opening.

4) The transoral procedure mask of claim 1, further comprising a hollow conical protrusion attached to the side of the mask shell over the user's cheek, wherein the hollow conical protrusion is in fluid communication with the interior of the mask shell, wherein the oxygen tube is mounted to the apex of the hollow conical protrusion at the end of the hollow conical protrusion distal to the face mask shell, wherein the oxygen tube is in fluid communication with the interior of the face mask shell.

5) The transoral procedure mask of claim 2, further comprising a hollow conical protrusion attached to the side of the mask shell over the user's cheek, wherein the hollow conical protrusion is in fluid communication with the interior of the mask shell, wherein the oxygen tube is mounted to the apex of the hollow conical protrusion at the end of the hollow conical protrusion distal to the face mask shell, wherein the oxygen tube is in fluid communication with the interior of the face mask shell.

6) The transoral procedure mask of claim 3, further comprising a hollow conical protrusion attached to the side of the mask shell over the user's cheek, wherein the hollow conical protrusion is in fluid communication with the interior of the mask shell, wherein the oxygen tube is mounted to the apex of the hollow conical protrusion at the end of the hollow conical protrusion distal to the face mask shell, wherein the oxygen tube is in fluid communication with the interior of the face mask shell.

7) The transoral procedure mask of claim 4 wherein the second tube is connected to the side of the hollow conical protrusion and is in fluid communication with the interior of the hollow conical protrusion.

8) The transoral procedure mask of claim 5, wherein the second tube is connected to the side of the hollow conical protrusion and is in fluid communication with the interior of the hollow conical protrusion.

9) The transoral procedure mask of claim 2, further comprising a bite block, wherein the bite block comprises a passageway through the bite block with an area of at least 0.5 square inches that passes through the bite block, wherein the passageway opens into the user's mouth, wherein the passageway allows access for at least one of tools and instruments into the patient's mouth.

10) The transoral procedure mask of claim 7, further comprising a bite block, wherein the bite block comprises a passageway through the bite block with an area of at least 0.5 square inches that passes through the bite block, wherein the passageway opens into the user's mouth, wherein the passageway allows access for at least one of tools and instruments into the patient's mouth.

11) The transoral procedure mask of claim 8, further comprising a bite block, wherein the bite block comprises a passageway through the bite block with an area of at least 0.5 square inches that passes through the bite block, wherein the passageway opens into the user's mouth, wherein the passageway allows access for at least one of tools and instruments into the patient's mouth.

13) The transoral procedure mask of claim 9, wherein the face mask shell comprises a middle section surrounding the user's mouth and nose, wherein the middle section is constructed of a rigid polymer; wherein the face mask shell further comprises an outer section attached to the outer periphery of the middle section, wherein the outer section is constructed of a pliable polymer designed to better fit against and seal to a user's face.

14) The transoral procedure mask of claim 10, wherein the mask shell comprises a middle section surrounding the user's mouth and nose, wherein the middle section is constructed of a rigid polymer, wherein the conical protrusion is attached to the side of middle section of the mask; wherein the face mask shell further comprises an outer section attached to the outer periphery of the middle section, wherein the outer section is constructed of a pliable polymer designed to better fit against and seal to a user's face.

15) The transoral procedure mask of claim 11, wherein the mask shell comprises a middle section surrounding the user's mouth and nose, wherein the middle section is constructed of a rigid polymer, wherein the conical protrusion is attached to the side of middle section of the mask; wherein the face mask shell further comprises an outer section attached to the outer periphery of the middle section, wherein the outer section is constructed of a pliable polymer designed to better fit against and seal to a user's face.
